(19) United States
(12) Patent Application Publication
(10) Pub. No.: US 2009/0082806 A1
(43) Pub. Date: Mar. 26, 2009

(54) MENISCAL REPAIR SYSTEM

(75) Inventors: Hugh S. West, JR., Sandy, UT (US); Peter C. Miller, Largo, FL (US)

Correspondence Address: Workman Nydegger;1000 Eagle Gate Tower, 60 East South Temple, Salt Lake City, UT, 84111, US

(73) Assignee: HS West Investments, LLC, Sandy, UT (US)

(21) Appl. No.: 11/860,407

(22) Filed: Sep. 24, 2007

(51) Int. Cl. A61B 17/04

(52) U.S. Cl. 606/232; 606/233

(57) ABSTRACT

A meniscal repair system including a pair of elongate needles with a suture filament attached between respective proximal ends of the needles. Two implants are attached to the suture (e.g., molded thereon) between the needles so as to form a suture bridge between the implants. One implant may comprise a barbed shaft aligned with the suture, while the other may be in the form of a T-bar that can be deployed so as to be perpendicular to a portion of the suture filament. The system may further include one or more dilators (e.g., one or more suture knots or molded beads) disposed along the length of the suture filament to aid in passage of the implant after the associated needle.

### BACKGROUND OF THE INVENTION

1. The Field of the Invention

The present invention relates to systems and methods for use in repairing soft tissue (e.g., cartilage) tears, for example a meniscal tear formed in the meniscus of a patient's knee.

2. The Relevant Technology

It has been shown that fixation of meniscus traumas, like ruptures and lesions by suturing gives better results than the removal of traumatized meniscal tissue. However, existing repair techniques are often complicated and tedious and present risks for the patient. For example there is a risk that vessels and/or nerves may be damaged during the surgery. Therefore, development efforts have centered on providing meniscal lesion fixation devices and methods which may be used more safely and rapidly. Although various meniscal lesion fixation devices and methods have been developed, such devices and methods often require complicated surgical techniques or complex configurations of components, which can also lead to damage to the surrounding tissue. For example, such techniques and systems often leave tied knots, clips, or locking mechanisms on or near meniscal surfaces which are subject to rubbing against other tissues. The presence of such hard and/or sharp non-biological materials and/or components can result in further damage to surrounding tissue.

As such, there is a continuing need for meniscal repair systems and methods that may be accomplished with minimal invasion, which are rapid, easy to install, and which provide strong and safe fixation of a tissue tear.

### BRIEF SUMMARY OF THE PREFERRED EMBODIMENTS

In one embodiment, the present invention is directed to a meniscal repair system including a pair of elongate needles with a suture filament attached between respective proximal ends of the needles. Two implants are attached to the suture filament (e.g., molded thereon) between the needles so as to form a suture bridge between the implants. One implant may comprise a barbed shaft with a longitudinal axis aligned with the suture, while the other may be in the form of a T-bar that can be deployed by pivoting so as to be perpendicular to one end of the suture. Each needle is associated with one of the implants. The system is particularly configured for pulling the implants into place, rather than pushing the implants into place, which pulling provides for greater control during placement.

The system may further include one or more dilators (e.g., one or more suture knots, molded beads, and/or other means for dilating) disposed along the length of the suture filament. In one embodiment, a first plurality of spaced apart dilators is disposed along the suture filament between the first needle and the first soft tissue implant, and a second plurality of spaced apart dilators is disposed along the suture filament between the second needle and the second implant. The first plurality of dilators are advantageously arranged so as to be of progressively increasing size (i.e., a dilator of the first plurality of dilators having a smallest size is situated closest to a proximal end of the first needle). The second plurality of dilators may advantageously be configured in a similar manner so that the smallest dilator (e.g., having the smallest diameter or thickness) is situated closest to a proximal end of the second needle.

An alternative system also configured for pulling includes an implant with a fish-vertebrae type configuration in which the anchor implant may comprise separate sections, each section being molded or otherwise attached to a suture filament. An anteriorly disposed head section which includes an anterior head of a small cross-sectional area so as to be relative sharp, which allows easier penetration as the head section follows a needle into an opening. Such a system may include two implants and two needles, as the system described above, or it may only include a single needle and single implant connected by a suture filament that runs between the anteriorly located needle and the posteriorly located implant.

In a related method of use, one needle is used to pierce the meniscus from the inside of the patient's knee. The needle is initially pushed through and across the meniscal tear until the needle exits the skin out the back of the knee. The needle is then used to pull its associated implant into place on one end of the meniscal tear. Once one implant is in position, the other needle is pushed through the meniscus on the other side of the tear (so as to allow closing of the tear) and out the back of the knee. The needle is then pulled so as to pull the other implant into place. Pulling of the implants provides greater control to the practitioner as compared to trying to push the implants through the meniscal tear and into a desired position. The dilators (e.g., one or more knots within the suture filament and/or one or more beads or other shapes molded onto the suture) act to enlarge the passageway as the suture filament and dilator(s) are pulled through behind the needle in preparation for the implant. The presence of the dilators minimizes any tendency for the following implant to tear or otherwise damage adjacent tissue during pulling of the implant

### BRIEF DESCRIPTION OF THE DRAWINGS

To further clarify the above and other advantages and features of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of an exemplary meniscal repair system;

FIG. 2A illustrates a close up view of the first implant of the system of FIG. 1;

FIG. 2B illustrates a close up view of the first implant of FIG. 2B in a deployed, pivoted, T-bar configuration;

FIG. 3A illustrates a close up view of an alternative implant that may be used with a meniscal repair system;

FIG. 3B illustrates an alternative system including the implant of FIG. 3A.

FIG. 4A illustrates an inside-out method of using the system of FIG. 1 in which a first needle has been pushed through a meniscal tear and in which a first implant is being pulled into position;

FIG. 4B illustrates the first implant being pulled so that the first implant exits the external surface of the meniscus;

FIG. 4C illustrates tension having been applied to an opposite end of the suture filament so as to cause the first implant to pivot to a deployed, T-bar substantially perpendicular position;

FIG. 4D illustrates the meniscal tear being closed after the second needle has been pushed through the meniscal tear, and the second implant is being pulled into position; and

FIG. 4E illustrates the meniscal tear held in a closed position as a result of the anchoring action of the first and second implants and a suture bridge between the implants.

### DETAILED DESCRIPTION OF THE PREFERRED EMBODIMENTS

### I. Introduction

In one embodiment, the present invention is directed to a meniscal repair system including a pair of elongate needles with a suture filament attached between respective proximal ends of the needles. Two implants are attached to the suture filament (e.g., molded thereon) between the needles so as to form a suture bridge between the implants. One implant may comprise a barbed shaft with a longitudinal axis aligned with the suture, while the other may be in the form of a T-bar that can be deployed by pivoting so as to be perpendicular to one end of the suture. The system may further include one or more dilators (e.g., one or more suture knots, molded beads, and/or other means for dilating) disposed along the length of the suture filament. Each needle is associated with one of the implants.

In one method of use, one needle is used to pierce the meniscus from the inside of the patient's knee. The needle is initially pushed through and across the meniscal tear until the needle exits the skin out the back of the knee. The needle is then used to pull its associated implant into place on one end of the meniscal tear. Once one implant is in position, the other needle is pushed through the meniscus on the other side of the tear (so as to allow closing of the tear) and out the back of the knee. The needle is then pulled so as to pull the other implant into place. Pulling of the implants provides greater control to the practitioner as compared to trying to push the implants through the meniscal tear and into a desired position. The dilators (e.g., one or more knots within the suture filament and/or one or more beads or other shapes molded onto the suture) act to enlarge the passageway as the suture filament and dilator(s) are pulled through behind the needle in preparation for the implant. The presence of the dilators minimizes any tendency for the following implant to tear or otherwise damage adjacent tissue during pulling of the implant.

### II. Exemplary Systems

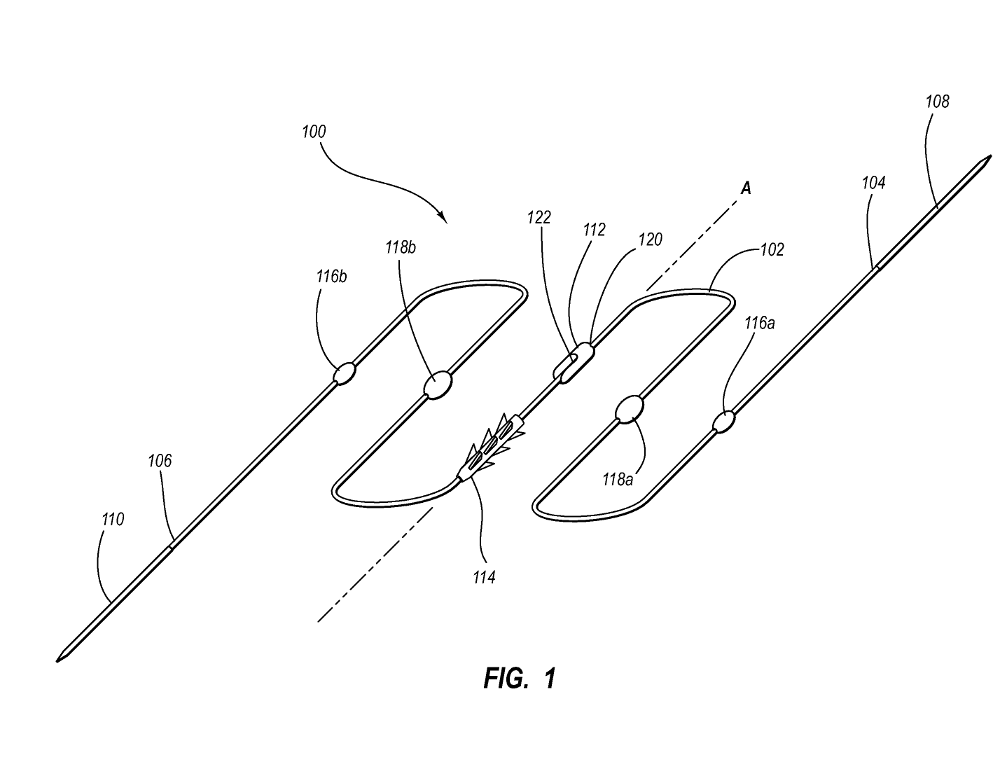

FIG. 1 illustrates an exemplary meniscal repair system 100 including an elongate suture filament 102 having a first end 104 and a second end 106. A proximal end of first needle 108 is attached to first end 104 of filament 102, while a proximal end of second needle 110 is attached to second end 106 of filament 102. A first soft tissue implant 112 is attached to suture filament 102 at a desired first position along the suture, while a second soft tissue implant 114 is attached to suture filament 102 at a different, second position along the suture filament 102. The second position of second implant 114 is located between the position of first implant 112 and the second end 106 of suture filament 102. First needle 108 is associated with first implant 112 and second needle 110 is associated with second implant 114, as in positioning, first needle 108 is used to pull implant 112 into position, and second needle 110 is used to pull second implant 114 into position.

The illustrated system 100 further includes a plurality of dilators affixed to suture filament 102 at spaced apart locations along suture filament 102 between each needle and its associated implant. For example, a pair of dilator beads 116a and 118a are disposed along suture filament 102 between needle 108 and implant 112. The two dilator beads are arranged so that first dilator 116a, which is smaller in size (i.e., it has a smaller maximum thickness) than dilator 118a, is disposed closer to needle 108 than second dilator 118a. Similarly, another first dilator 116b, which is smaller in size than second dilator 118b, is disposed closer to needle 110 than second dilator 118b.

Although illustrated with pairs of dilators between each needle and each implant, an alternative embodiment may include fewer (e.g., 0-3) or more dilators. As described above, in embodiments including two or more dilators between each needle and each implant, the dilators may preferably be arranged so as to be progressively larger in size with the smallest dilator nearest the associated needle and the largest dilator being situated closest to the associated implant. This allows passage of the smallest dilator behind the associated needle, which is then followed by progressively larger dilators so as to enlarge the passageway in preparation for the associated implant. Although illustrated as beads which may be molded onto suture 102, dilators having different shapes (e.g., spheres) or other structures (e.g., one or more knots tied into suture 102, or other dilating means) may be used. In one embodiment a knot may be formed into suture 102, after which a plastic dilator bead is molded over the knot. Beads having a smooth oval like shape (as illustrated) may be particularly preferred over other shapes or structures as they act to gently enlarge the passageway in preparation for passage of the implant as a result of their smooth outer surface and oval shape (i.e., an oval shape oriented as illustrated presents a more gentle slope as the dilator is pulled into the opening relative to a sphere).

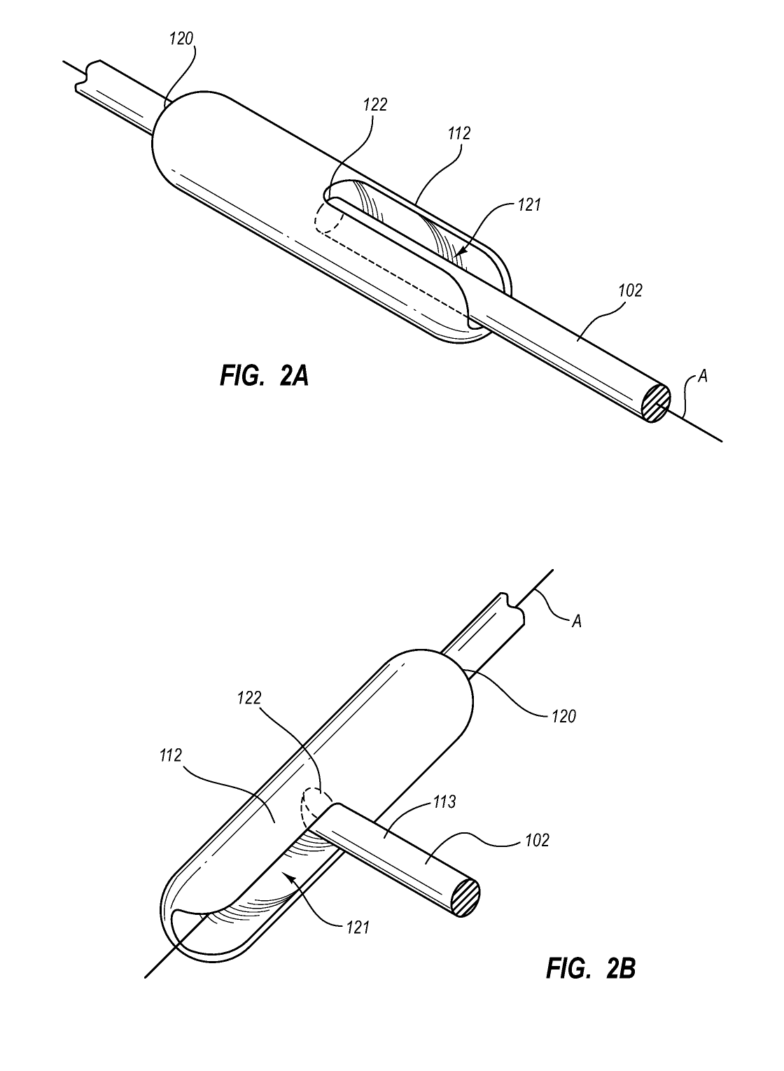

As perhaps best seen in FIGS. 2A and 2B, in the illustrated embodiment, first implant 112 comprises a deployable T-bar while second implant 114 comprises a barbed arrow shaped anchor (FIG. 1). Implant 112 is elongated so as to define a longitudinal axis A. Suture 102 is attached to first implant 112 at a first location 120 along axis A (at an end of implant 112) and at a second location 122 which is not at an opposite end of implant 112 along Axis A, but is spaced apart from an opposite end of implant 112 so as to allow implant 112 to pivot to a T-bar configuration. For example, in the illustrated embodiment location 122 is at about the mid-point along the length of implant 112. The result of such attachment of implant 112 to suture filament 102 is that implant 112 is pivotally attached to suture filament 102. Illustrated embodiment of implant 112 further includes a longitudinal groove 121 formed therein and configured to receive suture filament 102 when both illustrated sides of the suture filament are aligned with longitudinal axis A of implant 112, as shown in FIG. 2A. Upon application of tension to portion 113 of suture filament 102 attached at location 122, implant 112 deploys by pivoting so as to create a T-bar configuration relative to portion 113 of suture filament 102 where axis A of implant 112 is substantially perpendicular to portion 113 of suture filament 102 adjacent to location 122. The attachment of suture 102 at location 122 (e.g., at or near a mid point along a length of implant 112) along with groove 121 allows a pivoting deployment of implant 112 as illustrated.

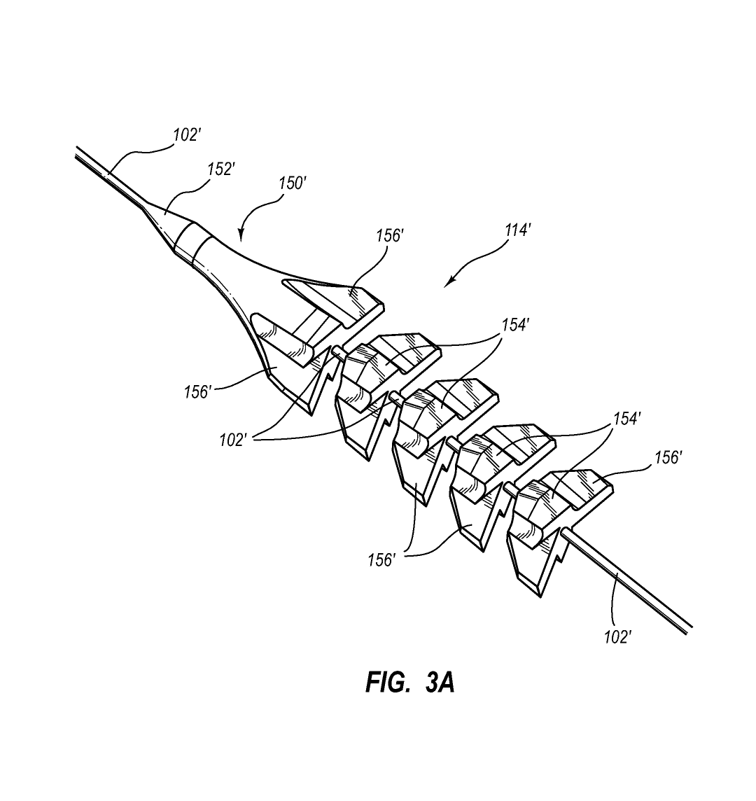

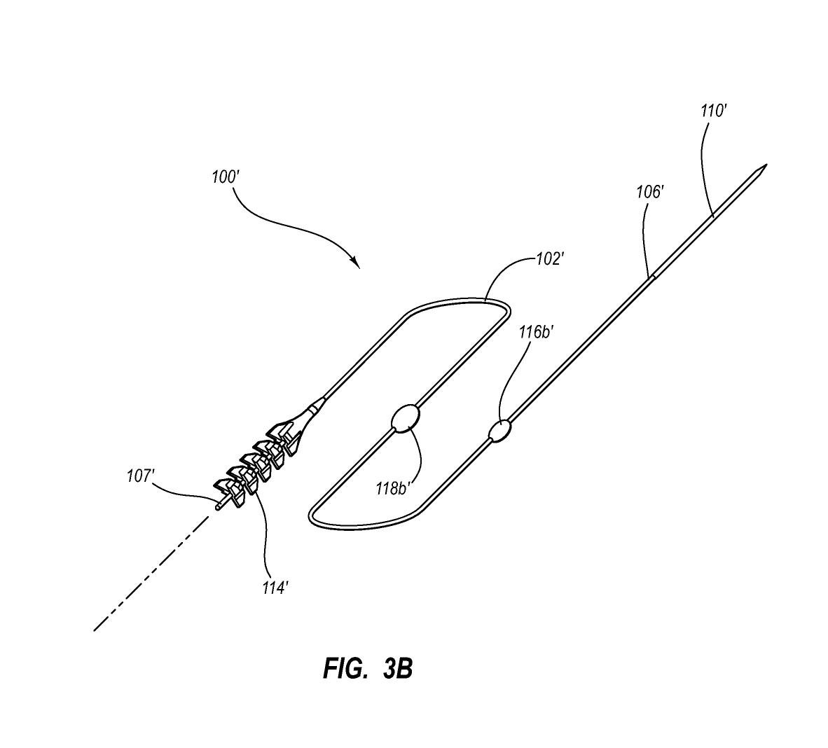

FIG. 3A illustrates an alternative implant 114′ that is particularly well suited for use with a needle so as to pull (rather than push) the implant 114′ into the meniscal tissue at a desired location. Implant 114′ is of a fish-vertebrae type in which the anchor implant 114′ may comprise separate sections, each section being molded or otherwise attached to suture filament 102′. In the illustrated embodiment, implant 114′ includes a head section 150′ which includes an anterior head of a small cross-sectional area (so as to be relative sharp, which allows easier penetration as the head section 150′ follows a needle (e.g., needle 110′ of FIG. 3B) into an opening. Although illustrated as being substantially pointed, the anterior head 152′ of section 150′ may include other shapes (e.g., rounded), although any alternative preferred configuration will include an anterior head that is of a relatively small cross-sectional area so as to aid in following a needle through the opening defined in the meniscal tissue by the needle.

Head section 150′ is followed by one or more posteriorly located tail sections 154′ which may be molded or otherwise attached to suture filament 102′ so as to be spaced apart. Illustrated tail sections 154′ each include a plurality of fin-like barbs 156′ to aid in securing implant 114′ within the meniscal tissue. The illustrated configuration including a head section 150′ and one or more tail sections 154′, wherein each section is spaced apart from the preceding section by a length of suture filament 102′ provides excellent flexibility to anchor implant 114′ as a result of its fish-vertebrae like configuration. Such flexibility is helpful when placing the implant, and is not detrimental during a pulling placement, while such flexibility might be impractical if attempting to position such an implant by pushing. Although illustrated as only being connected by suture filament 102′, an alternative embodiment may be configured such that each section of anchor implant 114′ is attached by a relatively thin section (e.g., molded over filament 102′), between barb fins 156′, although in such an embodiment the individual sections are still substantially spaced apart so as to provide similar flexibility along the longitudinal axis of the implant 114′ (i.e., implant 114′ can be easily flexed along its longitudinal axis).

FIG. 3B illustrates an alternative system 100′ which is somewhat similar to system 100 of FIG. 1, but which only includes a single needle 110′, optional dilators 116b′, 118b′, and a single fish-vertebrae type flexible pulling placement implant 114′, as illustrated and described above in conjunction with FIG. 3A. Needle 110′ is located at a first end 106′ of suture filament 102′, while flexible implant 114′ is located at second end 107′ of suture filament 102′. In use, system 100′ is used in a similar manner as system 100, except only a single implant 114′ is placed for each system so as to close all or a portion of a meniscal tear. The needle 110′ is used to penetrate through meniscal tissue at a desired location (e.g., at one side of a meniscal tear), and then any optional dilators and implant 114′ are pulled through along the path defined by needle 110′ and suture filament 102′. Implant 114′ is pulled into position so as to close the meniscal tear. Additional systems 100′ may be used along the length of the meniscal tear, if needed. Although illustrated with system 100′, implant 114′ may of course also be used with a system similar to system 100 (e.g., by replacing second implant 114 with implant 114′).

### III. Exemplary Methods of Use

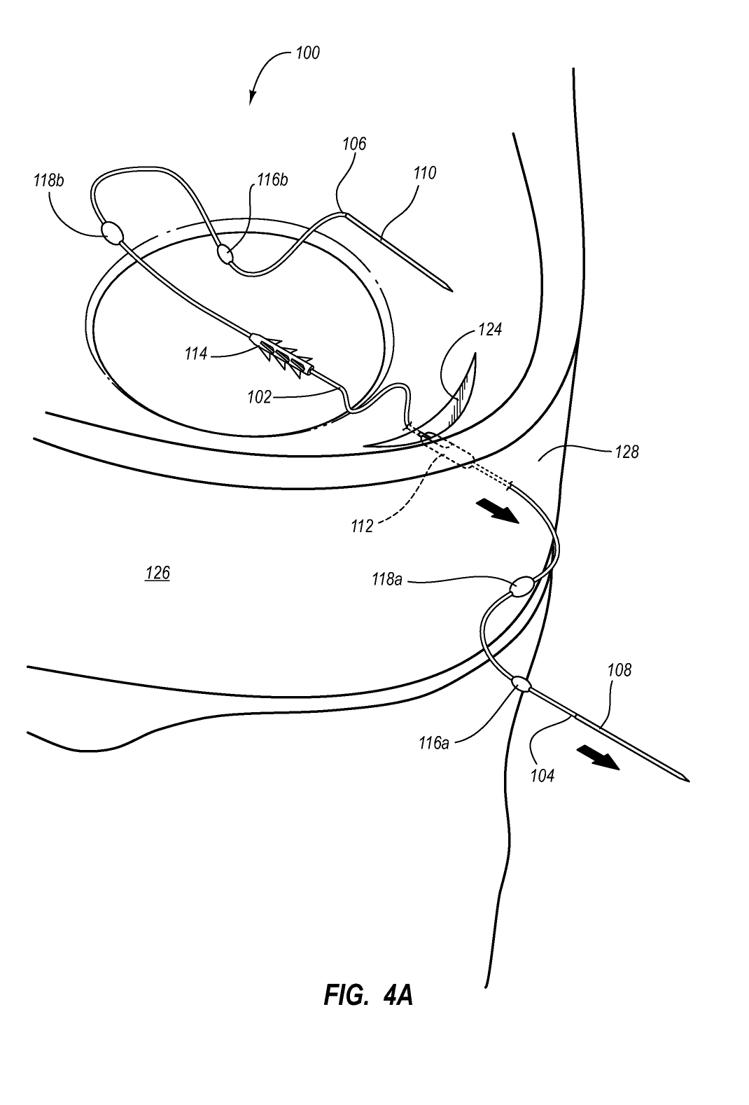

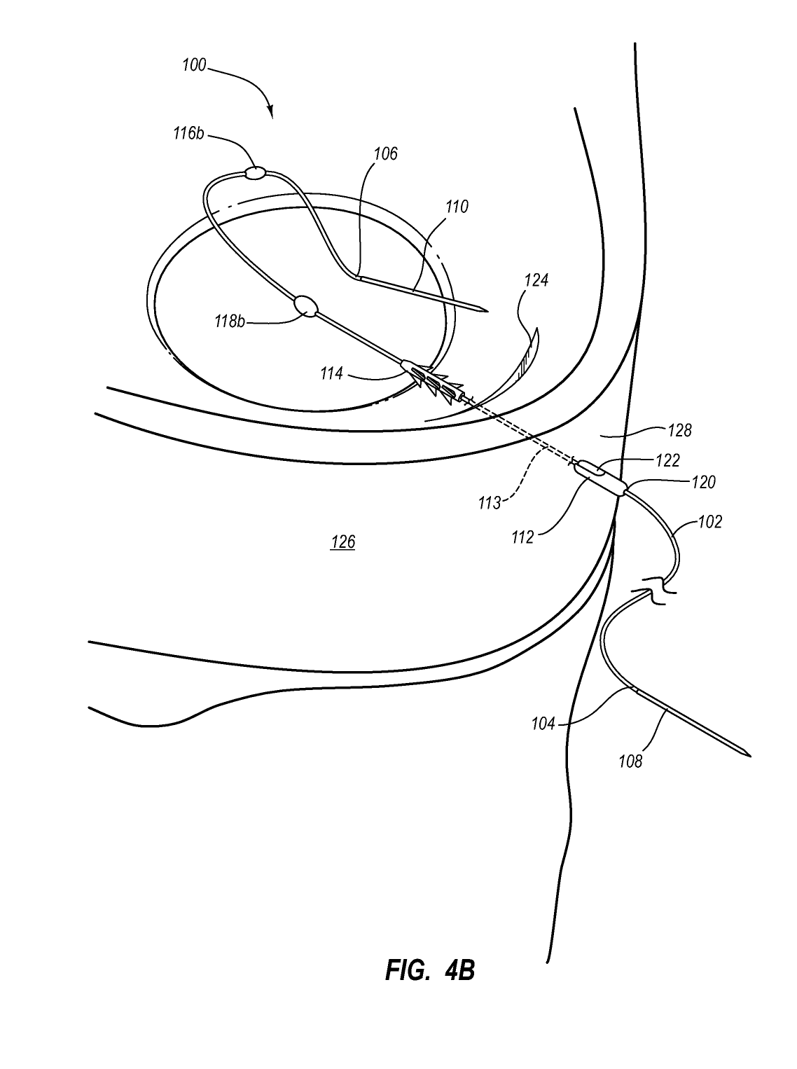

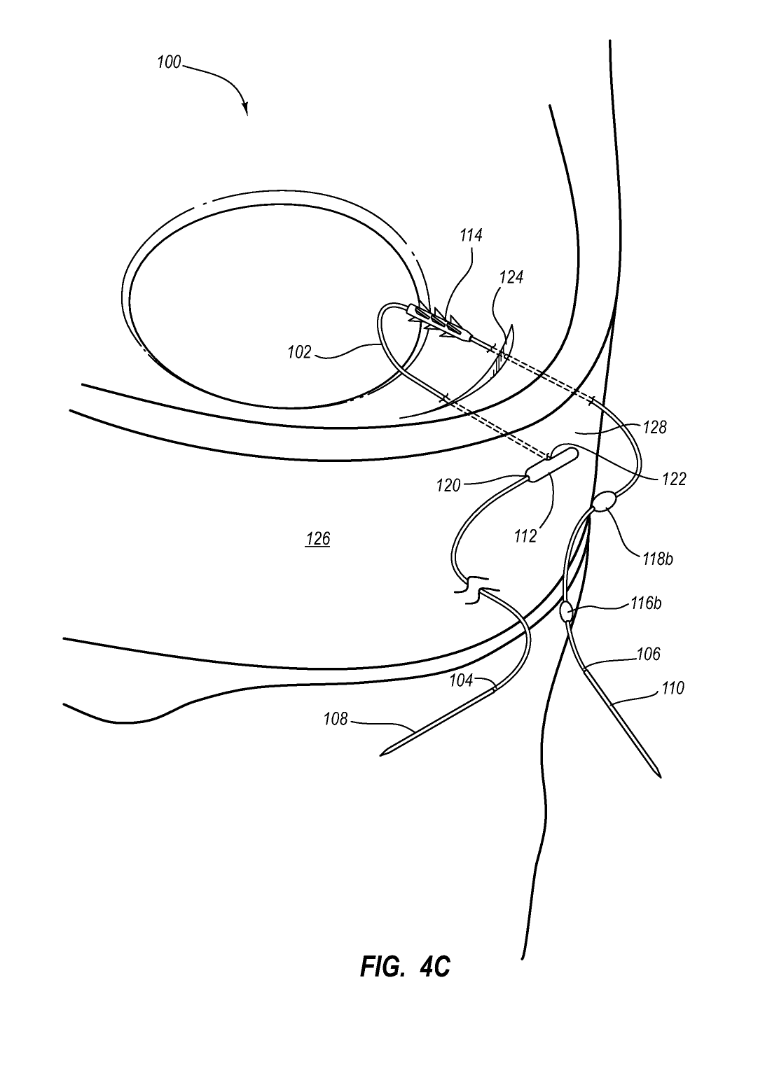

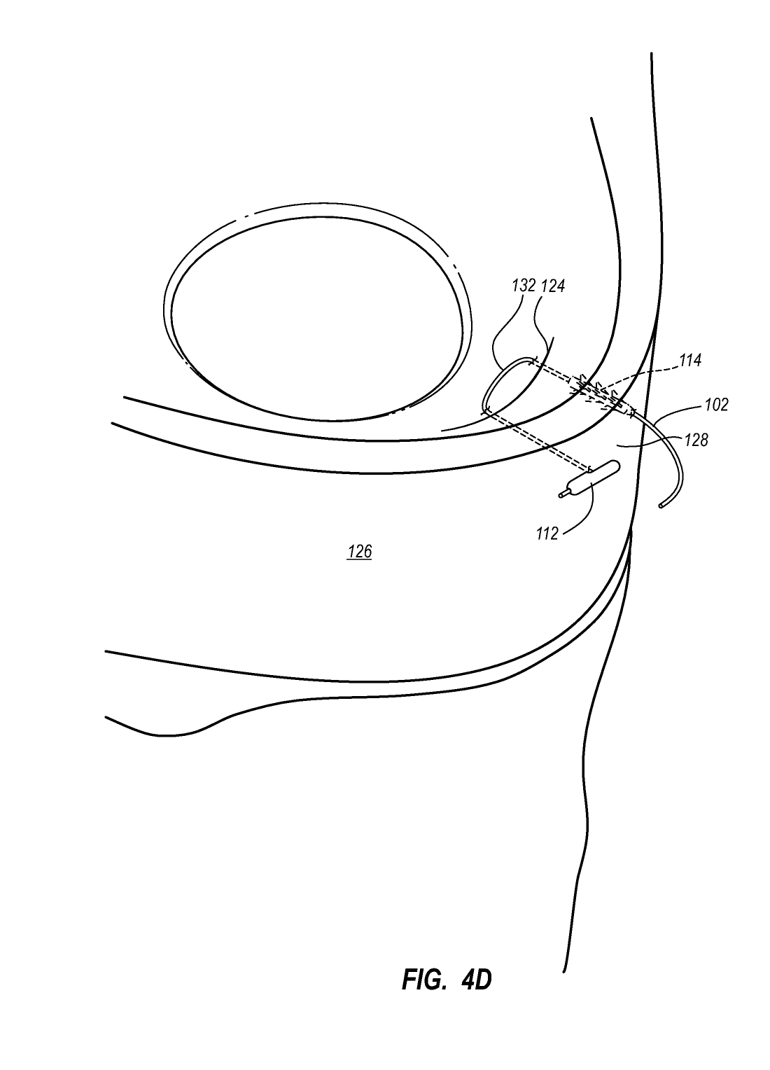

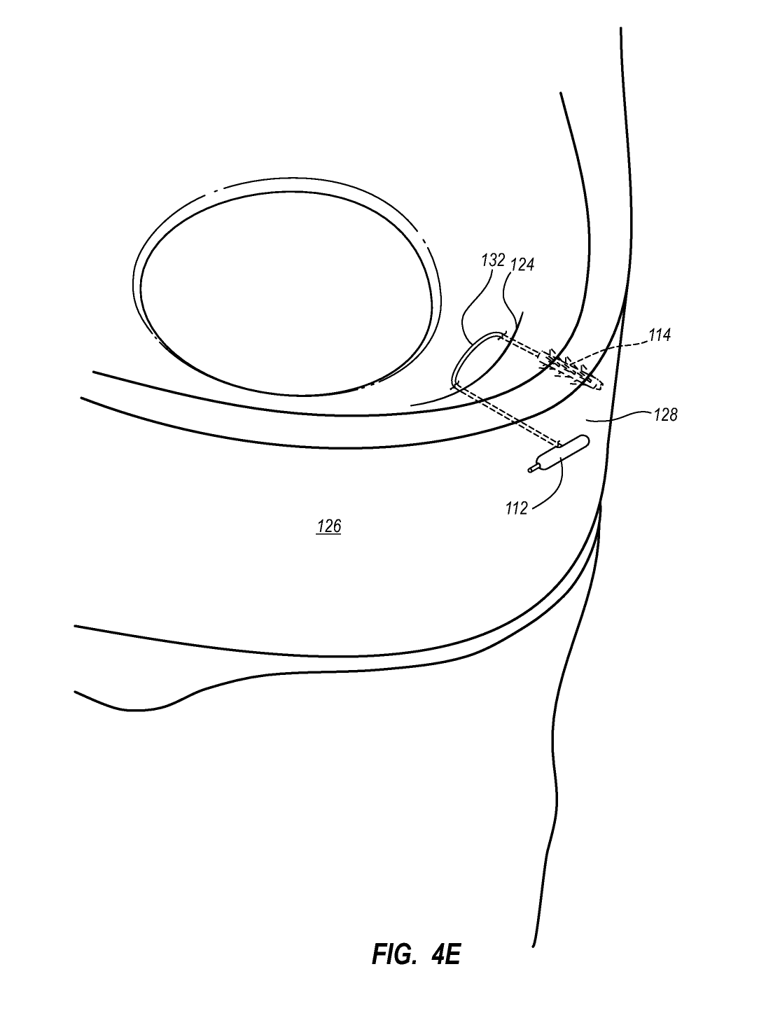

FIGS. 4A-4E illustrate an exemplary method of using system 100 to close a meniscal tear 124 within meniscus 126 using an inside-out technique. First needle 108 is pushed through meniscal tear 124 from the inside of the patient's knee towards the outside so that needle 108 exits through an exterior surface 128 of meniscus 126. A cannula (not shown) may be provided for guiding the needle as it is pushed through tear 124. Once outside the meniscus and skin (not shown), the practitioner pulls needle 108 and/or suture filament end 104 so as to pull dilators 116a, 118a and first implant 112 through the passageway defined by needle 108 and suture 102. First and second dilators 116a and 118a respectively follow behind needle 108 so as to enlarge the passageway through which first implant 112 follows. FIG. 4A shows the system in a configuration where needle 108 and dilators 116a and 116b have exited exterior surface 128 of meniscus 126 and in which first implant 112 is being pulled through meniscal tear 124. Pulling implant 112 into position as opposed to pushing the implant allows the practitioner to achieve a greater degree of control and direction as movement of first implant 112 progresses through tear 112. Using needle 108, suture 102 and dilators 116a and 118a to first open and define a passageway which implant 112 may follow advantageously allows for such increased control.

As shown in FIG. 4B, the practitioner continues to pull end 104 of suture filament 102 until first implant 112 exits the external surface 128 of meniscus 126. Once in this position, the practitioner may apply tension on portion 113 of suture filament 112 by pulling, for example, second needle 110 or end 106 of suture filament 102. Application of such tension causes T-bar implant 112 to pivot to a position as shown in FIG. 4C. Any additional tension applied to end 106 of suture filament 102 will ensure that T-bar implant 112 is deployed snugly against exterior surface 128 of meniscus 126 so as to anchor suture 102 to exterior surface 128.

Second needle 110 may then be pushed through meniscal tear 124 at a second location which is spaced apart from the location through which needle 108 was pushed. Once second needle 110 exits the exterior surface 128 of the meniscus and is pulled through the skin at the back of the knee, needle 110 may be pulled so as to pull and guide dilators 116b and 118b through the passageway defined by needle 110 and suture filament 102. As needle 110 and/or suture filament end 106 continues to be pulled, second implant 114, for example illustrated as an arrow shaped anchor including retention barbs formed thereon, is pulled through the passageway previously defined by needle 110. Because implant 114 is pulled into position rather than pushed, the practitioner has a greater degree of control over movement and positioning of implant 114, as needle 110 (which is much smaller and sharper relative to implant 114) is first pressed through meniscal tear 124 at a desired location so as to exit exterior surface 128 at a desired location. Once the practitioner is satisfied with the positioning and pathway defined by needle 110, the dilators 116b, 118b and second implant 114 are pulled through the same passageway, as shown in FIG. 4C, pulling implant 114 exactly into a desired position.

As second implant 114 is pulled into meniscal tear 124 the walls on either side of the tear are pressed and pulled together by the action of the retention barbs formed on implant 114 and suture 102. As shown in FIG. 4D, once second implant 114 passes completely through meniscal tear 124, the tear surfaces are brought together so as to close the tear. Implants 112 and 114 are connected by suture bridge 132 (which spans the tear), and tension on bridge 132 aids in forcing tear 124 closed so that healing may occur. In the illustrated embodiment, implant 114 remains entirely within meniscus 126 and may be formed of a bioabsorbable material capable of being absorbed by the body over time (e.g., polylactic and/or polyglycolic acid). First implant 112 and suture filament 102 may be formed of a similar bioabsorbable material. Each end of suture 102 including needles 108, 110 and dilators 116a, 118a and 116b, 118b may be cut off, leaving only implants 112 114, and suture bridge 132 in place.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A meniscal repair system, comprising:
an elongate suture filament having a first end and a second end;
a first soft tissue implant attached to said suture filament at a first position along a length of said suture filament;
a second soft tissue implant attached to said suture filament at a second position along the length of said suture filament, said second position being disposed between said first position and said second end of said suture filament;
a first needle, attached to said first end of said suture filament, for penetrating through meniscal tissue and drawing said first soft tissue implant through the meniscal tissue during use; and
a second needle, attached to said second end of said suture filament, for penetrating through meniscal tissue and drawing said second soft tissue implant through the meniscal tissue during use.

2. A system as recited in claim 1, further comprising one or more dilators affixed to said suture filament at one or more locations between one of said needles and an associated soft tissue implant.

3. A system as recited in claim 2, wherein said one or more of said dilators are affixed at one or more locations between said first needle and said first soft tissue implant and at one or more of said dilators are affixed at one or more locations between said second needle and said second soft tissue implant.

4. A system as recited in claim 2, wherein said one or more dilators comprise knots formed in said suture filament.

5. A system as recited in claim 2, wherein said one or more dilators comprise one or more beads molded onto said suture filament.

6. A system as recited in claim 1, further comprising:
a first plurality of spaced apart dilator beads molded onto said suture filament between said first needle and said first soft tissue implant, said first plurality of beads being of progressively increasing size so that a dilator bead of said first plurality of beads having a smallest size is situated closest to said proximal end of said first needle; and
a second plurality of spaced apart dilator beads molded onto said suture filament between said second needle and said second soft tissue implant, said second plurality of beads being of progressively increasing size so that a dilator bead of said second plurality of beads having a smallest size is situated closest to said proximal end of said second needle.

7. A system as recited in claim 1, wherein said first and second implants are elongated so as to each define a longitudinal axis, said second implant being attached to said suture filament at two locations, each location being at an opposite end of said second implant along said longitudinal axis of said second implant.

8. A system as recited in claim 7, wherein said first soft tissue implant is attached to said suture filament at two locations, one location being at one end of said first implant along said longitudinal axis of said first implant and the other of said two locations being at or near a mid point along a length of said first implant such that said first soft tissue implant is pivotably attached to said suture filament whereby it may assume an orientation that is substantially perpendicular to a portion of said suture filament.

9. A system as recited in claim 7, wherein said second soft tissue implant includes a plurality of laterally projecting retention barbs.

10. A meniscal repair system, comprising:
an elongate suture filament having a first end and a second end;
a first soft tissue implant comprising a T-bar having a longitudinal axis, said first implant being attached to said suture filament at a first position along a length of said suture filament, said first implant being attached to said suture filament at two locations, one location being at one end of said first implant along said longitudinal axis of said first implant and the other of said two locations being at or near a mid-point along a length of said implant such that said first soft tissue implant is pivotably attached to said suture filament whereby it may assume an orientation that is substantially perpendicular to a portion of said suture filament;
a second soft tissue implant comprising an anchor including a plurality of laterally projecting retention barbs, said second implant being attached to said suture filament at a second position along the length of said suture filament, said second position being disposed between said first position and said second end of said suture filament;
a first needle, attached to said first end of said suture filament, for penetrating through meniscal tissue and drawing said first soft tissue implant through the meniscal tissue during use;
a second needle, attached to said second end of said suture filament, for penetrating through meniscal tissue and drawing said second soft tissue implant through the meniscal tissue during use;
a first plurality of spaced apart dilators disposed on said suture filament between said first needle and said first soft tissue implant, said first plurality of dilators being of progressively increasing size so that a dilator of said first plurality of dilators having a smallest size is situated closest to said proximal end of said first needle; and
a second plurality of spaced apart dilators disposed on said suture filament between said second needle and said second soft tissue implant, said second plurality of dilators being of progressively increasing size so that a dilator of said second plurality of dilators having a smallest size is situated closest to said proximal end of said second needle.

11. A system as recited in claim 10, wherein said second implant is attached to said suture filament at two locations, each location being at an opposite end of said second implant along said longitudinal axis of said second implant.

12. A meniscal repair system, comprising:
an elongate suture filament having an anterior first end and a posterior second end;
a soft tissue implant attached to said suture filament at or near the posterior second end of said suture filament, said soft tissue implant comprising a head section and one or more substantially spaced apart tail sections, each section being molded or otherwise attached to the suture filament such that the implant is flexible along a longitudinal axis of the implant; and
a needle attached to said anterior first end of said suture filament, said needle being configured for penetrating through meniscal tissue and drawing said soft tissue implant through the meniscal tissue during use.

13. A method of repairing a meniscal tear comprising:
pushing a first needle from inside a patient's knee through a meniscal tear so that said first needle exits skin surrounding a patient's knee, a proximal end of said first needle being attached to a suture filament;
pulling said first needle and/or said suture filament so as to draw a first soft tissue implant attached along said suture filament into a position on one side of said meniscal tear;
pushing a second needle from inside a patient's knee through said meniscal tear so that said second needle exits skin surrounding a patient's knee, a proximal end of said second needle being attached to said suture filament; and
pulling said second needle and/or said suture filament so as to draw a second soft tissue implant attached along said suture filament through a passageway formed by said second needle and said suture filament.

14. A method as recited in claim 13, wherein said first implant is attached to said suture filament at two locations, one location being at one end of said implant and the other of said two locations being at or near a mid-point along a length of said first implant such that said first soft tissue implant is pivotably attached to said suture filament, further comprising the step of applying tension to a portion of the suture filament whereby the first implant assumes an orientation that is substantially perpendicular to said portion of the suture filament.

15. A method as recited in claim 14, wherein said first implant comprises a T-bar.

16. A method as recited in claim 13, wherein one or more dilators are disposed along said suture filament between said first needle and said first implant and between said second needle and said second implant, the one or more dilators enlarging a passageway formed by said needles and said suture filament in preparation for passage of said implants.

17. A method as recited in claim 16, wherein one or more dilators comprise one or more knots formed in said suture filament.

18. A method as recited in claim 16, wherein one or more dilators comprise one or more beads molded onto said suture filament.

19. A method as recited in claim 13, wherein:
a first plurality of spaced apart dilator beads are molded onto said suture filament between said first needle and said first soft tissue implant, said first plurality of beads being of progressively increasing size so that a dilator bead of said first plurality of beads having a smallest size is situated closest to said proximal end of said first needle, the first plurality of dilator beads enlarging a passageway formed by said first needle and said suture filament in preparation for passage of said first implant; and
a second plurality of spaced apart dilator beads are molded onto said suture filament between said second needle and said second soft tissue implant, said second plurality of beads being of progressively increasing size so that a dilator bead of said second plurality of beads having a smallest size is situated closest to said proximal end of said second needle the second plurality of dilator beads enlarging a passageway formed by said second needle and said suture filament in preparation for passage of said second implant.

20. A method as recited in claim 13, wherein said first and second implants are elongated so as to each define a longitudinal axis, said second implant being attached to said suture filament at two locations, each location being at an opposite end of said second implant along said longitudinal axis of said second implant.

21. A method as recited in claim 20, wherein said second soft tissue implant includes a plurality of laterally projecting retention barbs.
